(19) United States
(12) Patent Application Publication
(10) Pub. No.: US 2013/0253332 A1
(43) Pub. Date: Sep. 26, 2013

(54) TISSUE INTERFACE SYSTEMS FOR APPLICATION OF OPTICAL SIGNALS INTO TISSUE OF A PATIENT

(71) Applicants: Sarah Hayman, Boulder, CO (US); Mark Su, Boulder, CO (US)

(72) Inventors: Sarah Hayman, Boulder, CO (US); Mark Su, Boulder, CO (US)

(73) Assignee: Nellcor Puritan Bennett LLC, Boulder, CO (US)

(21) Appl. No.: 13/424,814

(22) Filed: Mar. 20, 2012

(51) Int. Cl. A61B 6/08

(52) U.S. Cl. 600/476

(57) ABSTRACT

Systems and methods for applying optical signals into tissue of a patient are provided herein. In one example, a tissue interface system for applying optical signals to tissue of a patient is provided. The tissue interface system includes a tissue interface pad configured to apply the optical signals carried by at least one optical source into the tissue, and a pressurized volume configured to apply pressure to the tissue interface pad to couple a portion of the tissue interface pad to the tissue.

### TECHNICAL FIELD

Aspects of the disclosure are related to the field of medical devices, and in particular, tissue interface systems for application of optical signals into tissue of a patient and optical measurement of physiological parameters of blood and tissue.

### TECHNICAL BACKGROUND

Various devices, such as pulse oximetry devices or photon density wave (PDW) devices, can measure parameters of blood or tissue in a patient, such as heart rate and oxygen saturation of hemoglobin, among other parameters. These devices are non-invasive measurement devices, typically employing solid-state lighting elements, such as light-emitting diodes (LEDs) or solid state lasers, to introduce light into the tissue of a patient. The light is then detected and analyzed to determine the parameters of the blood flow in the patient.

However, consistent application and detection of the light or other optical signals into the tissue of the patient can be difficult to achieve. For example, conventional devices typically include a hinged spring mechanism to clamp over a finger of a patient. These spring clamp devices are subject to patient-specific noise and inconsistencies which limits the accuracy of such devices. For example, the size of the tissue under measurement can vary from one patient to another, such as in examples of finger-based measurements. Clamp-style devices are thus typically limited in the ranges of patient tissue sizes, and thus cannot provide a consistent application of the optical signals into the tissue due to these varying tissue sizes.

In further examples, measurement and processing systems are located remotely from various optical elements used for interfacing optical signals with the tissue of the patient. This configuration can provide some patient mobility by using a flexible fiber optic cable between the equipment. However, having a long cable can introduce errors into the measurement and subsequent processing of the optical signals due to various mechanical stresses and tensions due to the long cables.

### Overview

Systems and methods for applying optical signals into tissue of a patient are provided herein. In one example, a tissue interface system for applying optical signals to tissue of a patient is provided. The tissue interface system includes a tissue interface pad configured to apply the optical signals carried by at least one optical source into the tissue, and a pressurized volume configured to apply pressure to the tissue interface pad to couple a portion of the tissue interface pad to the tissue.

In a second example, a method for applying optical signals to tissue of a patient is provided. The method includes applying the optical signals carried by at least one optical fiber into the tissue by a tissue interface pad, and applying a pressure from a pressurized volume to the tissue interface pad to couple a portion of the tissue interface pad to the tissue.

This Overview is provided to introduce a selection of concepts in a simplified form that are further described below in the Detailed Description. It should be understood that this Overview is not intended to identify key features or essential features of the claimed subject matter, nor is it intended to be used to limit the scope of the claimed subject matter.

### BRIEF DESCRIPTION OF THE DRAWINGS

FIG. 1 is a system diagram illustrating a system for applying optical signals to tissue of a patient.

FIG. 2 is a flow diagram illustrating a method of operation of a system for applying optical signals to tissue of a patient.

FIG. 3 is a system diagram illustrating a system for applying optical signals to tissue of a patient.

FIG. 4 is a system diagram illustrating a system for applying optical signals to tissue of a patient.

FIG. 5 is a system diagram illustrating a system for applying optical signals to tissue of a patient.

FIG. 6 is a system diagram illustrating a system for applying optical signals to tissue of a patient.

### DETAILED DESCRIPTION

Various physiological parameters of tissue and blood of a patient can be determined non-invasively, such as optically. In one example, optical signals introduced into the tissue of the patient are modulated according to a high-frequency modulation signal to create a photon density wave (PDW) optical signal in the tissue undergoing measurement. Due to the interaction between the tissue or blood and the PDW optical signal, various characteristics of the PDW optical signal can be affected, such as through scattering or propagation by various components of the tissue and blood. The various physiological parameters can include any parameter associated with the blood or tissue of the patient, such as regional oxygen saturation (rSO2), arterial oxygen saturation (SpO2), heart rate, lipid concentrations, among other parameters, including combinations thereof.

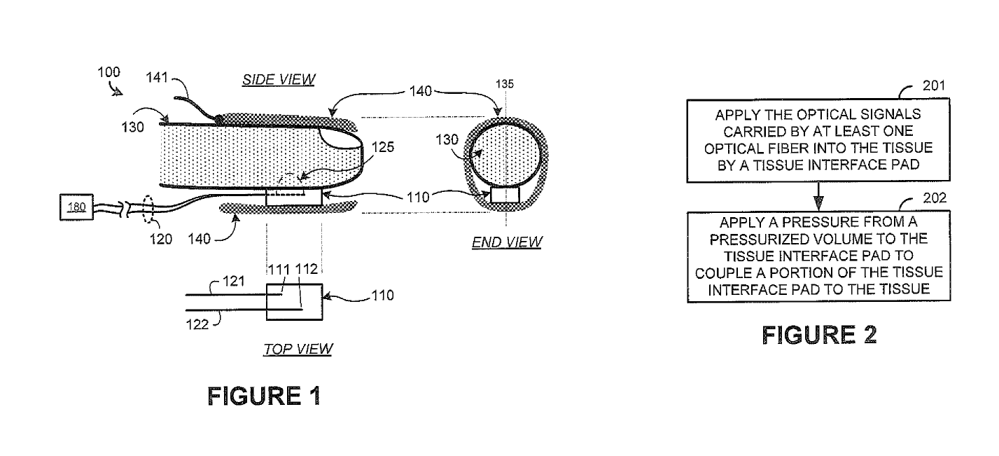

As a first example of a system for applying optical signals to tissue of a patient, FIG. 1 is presented. FIG. 1 illustrates system 100, which includes tissue interface pad 110, optical cable 120, tissue 130, and pressurized volume 140. A top view, end view, and side view of system 100 are included in FIG. 1 to highlight the various elements of system 100. The end view is sectioned at section cut 135. It should be understood the dashed features of FIG. 1 are merely intended to highlight various elements of system 100, and are not intended to be exact wireframe representations of the elements of system 100; variations are possible.

In operation, optical signals generated by measurement system 180 are applied to tissue 130 for measurement of a physiological parameter, as indicated by optical signals 125. In this example, optical signals 125 are applied to tissue 130 via input optical fiber 121 terminated at location 111 of tissue interface pad 110, and optical signals 125 are detected through tissue 130 via output optical fiber 122 terminated at location 112 of tissue interface pad 110. Pressurized volume 140 is configured to apply a pressure to tissue interface pad 110 to couple at least a portion of tissue interface pad 110 to tissue 130.

FIG. 2 is a flow diagram illustrating a method of operation of system 100 for applying optical signals to tissue of a patient. The operations of FIG. 2 are referenced herein parenthetically. In FIG. 2, tissue interface pad 110 applies (201) optical signals 125 carried by at least one optical source into tissue 130. Tissue interface pad 110 couples to biological tissue, namely tissue 130, to allow for introduction of optical signals 125 into tissue 130. Tissue interface pad 110 also allows for receipt of optical signals propagated through tissue 130. Tissue interface pad 110 routes input optical fiber 121 carrying input optical signals 125 to first location 111 in tissue interface pad 110 via a first guide channel disposed within tissue interface pad 110. Tissue interface pad 110 routes output optical fiber 122 carrying received optical signals 125 to second location 112 in tissue interface pad 110 via a second guide channel disposed within tissue interface pad 110.

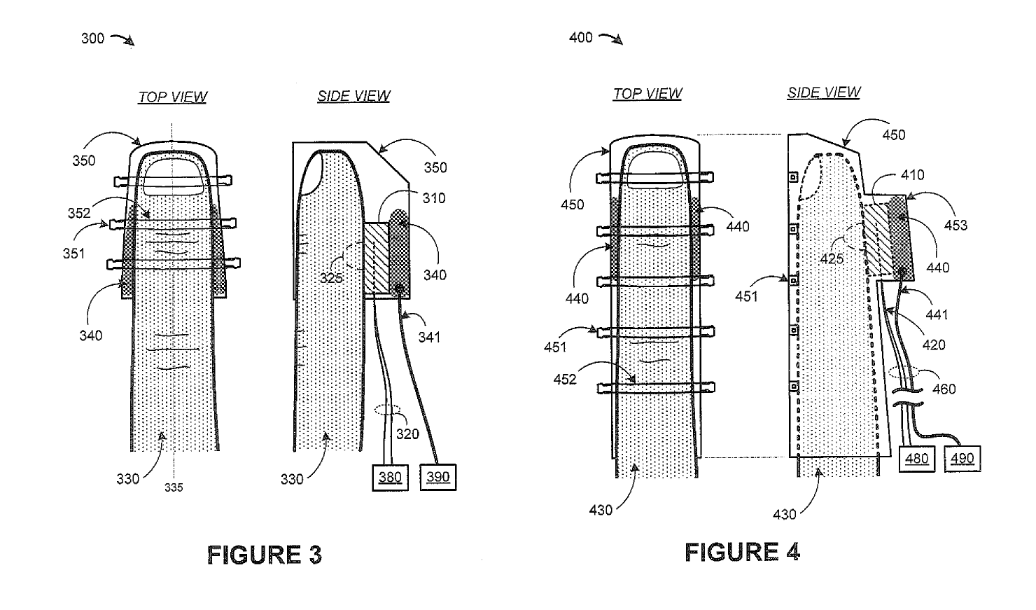

Pressurized volume 140 applies (202) a pressure to tissue interface pad 110 to couple a portion of tissue interface pad 110 to tissue 130. Although not required, FIG. 1 shows pressurized volume 140 receiving the pressure over pressure link 141. The pressure applied to tissue interface pad 110 allows for repeatable and controlled coupling of optical signals 125 to and from tissue 130 via tissue interface pad 110. The portion of tissue interface pad 110 which is coupled to tissue 130 includes at least locations 111-112 of tissue interface pad 110, which allows for optical signals carried by input optical fiber 121 to be introduced into tissue 130 and for optical signals propagated through tissue 130 to be received by output optical fiber 122. Although pressurized volume 140 is shown as an encircling pressure volume resembling a cuff in FIG. 1, other configurations can be employed, such as those illustrated in FIGS. 3-6.

Application of the pressure to pressurized volume 140 can occur through pressure link 141, although other configurations can be employed. Pressure link 141 can couple a pressure application device, such as a piston, syringe, pump, or other pressure application device to pressurized volume 140 via a tube or piping. Pressure sensors can be coupled to pressurized volume 140 to relay a presently applied pressure to a pressure control system or an operator for modification or monitoring of the pressure. In yet further examples, a quality of optical signals 125 is monitored, such as a magnitude of a pulsatile signal component detected over output fiber 122. The applied pressure may then be modified to ensure a desired quality of optical signals 125 in tissue 130. Upon receiving optical signals over optical fiber 122 after propagation through tissue 130, measurement system 180 may process the detected optical signals to determine various characteristics of the detected optical signals. Physiological parameters of the tissue and patient can then be identified based on the various characteristics of the detected optical signals.

Referring back to FIG. 1, tissue interface pad 110 comprises a physical structure having a surface that couples to biological tissue, namely tissue 130. The surface includes at least one optical signal emission point 111 and may include at least one optical signal detection point 112. Tissue interface pad 110 includes a mechanical arrangement to position and hold optical fibers 121-122 in a generally parallel arrangement to tissue 130. These mechanical arrangements can include grooves, channels, holes, snap-fit features, or other elements to route optical fibers 121-122 to a desired position in tissue interface pad 110. Tissue interface pad 110 may be comprised of plastic, foam, rubber, glass, metal, adhesive, or some other material, including combinations thereof. In some examples, tissue interface pad 110 is comprised of optically transmissive materials, such as optically transmissive plastic, glass, acrylic glass, polymethyl methacrylate (PMMA), or other materials, including combinations thereof. Optically transmissive adhesives can also be employed in tissue interface pad 110, such as to mate optical fibers 121-122 to optical interface elements of tissue interface pad 110. These optical adhesives can comprise Loctite 3321 or Norland 68 compositions which are cured using ultraviolet (UV) light. Other optically transmissive adhesives can be employed, including combinations thereof. In yet further examples, tissue interface pad 110 is thermally welded or otherwise adhered or attached to pressurized volume 140 to form an integrated tissue interface pad with pressurized volume.

Tissue 130 is shown in FIG. 1 as a finger of a patient. It should be understood that tissue 130 can be any tissue portion of a patient, such as a finger, toe, arm, leg, earlobe, forehead, or other tissue portion of a patient. In this example, tissue 130 is a portion of the tissue of a patient undergoing measurement of a physiological blood parameter. The wavelength of signals applied to the tissue can be selected based on many factors, such as optimized to a wavelength strongly absorbed by hemoglobin, lipids, proteins, or other tissue and blood components of tissue 130.

Pressurized volume 140 comprises an inflatable vessel for containing and applying a pressure to an external component, such as tissue interface pad 110. Examples of pressurized volume 140 include pressure cuffs, pressure pads, balloons, pistons, chambers, or other volumes which can receive and maintain a pressure via application of a pressurized fluid such as air to the volume. In some examples, pressurized volume 140 is integrated with a bandage configured to couple to tissue 130. The bandage can be made out of plastic, vinyl, PVC, plastic resin-filled paper, or other materials which allow the bandage to be flexible enough to conform to wrapping around tissue 130, such as a finger. The bandage is typically compatible to thermal welding of a thin and flexible cuff material. The cuff material can be in sheet form and thermally welded to the bandage such that the welding edges make an air-tight or pressure seal to form pressurized volume 140. It should be understood that although a wrapped or circular volume is discussed, other volumes styles can be employed. However, due to the pressure applied to tissue interface pad 110 and tissue 130, an equal and opposite force is typically also applied to an opposing side of tissue 130, such as by using a bandage/cuff that wraps around tissue 130 and applies the pressure around tissue 130 (as shown in FIG. 1), or by a structural or casing element coupled to tissue 130 and pressurized volume 140 which can include preloading elements. Further examples are discussed herein for FIGS. 3-6. Sizing of pressurized volume 140 or an associated bandage/cuff is typically driven by a size of tissue interface pad 110 to minimize overlap and movement of tissue 130 on application of the pressure and measurement. Velcro or adhesive can be placed on the bandage/cuff so pressurized volume 140 can be wrapped around tissue 130 and securely held from slippage or changing in orientation, as well as to allow for adjustment for different sizes of tissue 130. In yet further examples, tissue interface pad 110 is thermally welded or otherwise adhered or attached to pressurized volume 140 to form an integrated tissue interface pad with pressurized volume.

In alternative configurations, pressurized volume 140 comprises a static pressure element. The static pressure element can include a spring, foam pad, rubber pad, or other static pressure element which does not receive a pressure from an external source. Variations are possible, such as combination pressurized volumes which include both static pressure elements and dynamic pressure elements, such as an inflatable volume with a foam pad coupled thereto.

Measurement system 180 includes optical interfaces, digital processors, computer systems, microprocessors, circuitry, non-transient computer-readable media, user interfaces, or other processing devices or software systems, and may be distributed among multiple processing devices. Measurement system 180 may also include photon density wave (PDW) generation and measurement equipment, electrical to optical conversion circuitry and equipment, optical modulation equipment, and optical waveguide interface equipment. Measurement system 180 also includes laser elements such as a laser diode, solid-state laser, or other laser device, along with associated driving circuitry. Optical couplers, cabling, or attachments can be included to optically mate laser or detector elements to optical fibers 121-122.

Optical fibers 121-122 each comprise an optical waveguide, and each use glass, polymer, air, space, or some other material as the transport media for transmission of light, and can each include multimode fiber (MMF) or single mode fiber (SMF) materials. A sheath or loom can be employed to bundle optical fibers 121-122 together with further optical links for convenience, as indicated by optical cable 120. One end of each of optical fibers 121-122 mates with an associated optical driver or detector component of measurement system 180, and the other end of each of optical fibers 121-122 is configured to terminate in tissue interface pad 110 for optically interfacing with tissue 130. Various optical interfacing elements can be employed to optically couple optical signals carried by optical fibers 121-122 to tissue 130, such as prisms, reflective surfaces, refractive materials, or the like. Each of optical fibers 121-122 may include many different signals sharing the same associated link, as represented by the associated lines in FIG. 1, comprising channels, forward links, reverse links, user communications, overhead communications, frequencies, wavelengths, modulation frequencies, carriers, timeslots, spreading codes, logical transportation links, packets, or communication directions.

Also, although FIG. 1 illustrates two optical fibers 121-122, it should be understood that any number of input links and measurement links can be included, as well as any associated optical source and detector equipment. For example, tissue interface pad 110 may route many optical fibers to different physical locations on tissue 130, and these optical fibers can carry optical signals of different wavelengths. Alternatively, or in addition, tissue interface pad 110 may have measurement links positioned at different distances from input links or positioned over different anatomical structures. Also, although the optical source of FIG. 1 is shown as optical fiber 121 in this example, in further examples a direct light source can be included in tissue interface 110 and applied to tissue 130. Such direct light sources can include light-emitting diodes (LED), laser sources, or other signal sources, including combinations thereof.

The term ‘optical’ or ‘light’ is used herein for convenience. It should be understood that the applied and detected signals are not limited to visible light, and can comprise any photonic, electromagnetic, or energy signals, such as visible, infrared, ultraviolet, radio, x-ray, gamma, or other signals. Additionally, the use of optical fibers or optical cables herein is merely representative of a waveguide used for propagating signals between a transceiver and tissue of a patient. Suitable waveguides would be employed for different electromagnetic signal types.

FIG. 3 is a system diagram illustrating system 300 for applying optical signals to tissue 330 of a patient. System 300 includes tissue interface pad 310, optical cable 320, tissue 330, pressure cuff 340, casing 350, measurement system 380, and pressure system 390. A top view and a side view of system 300 are included in FIG. 3 to highlight the various elements of system 300. The side view is sectioned at section cut 335. It should be understood the dashed features of FIG. 3 are merely intended to highlight various elements of system 300, and are not intended to be exact wireframe representations of the elements of system 300; variations are possible.

Tissue 330 is shown as a finger of a patient undergoing measurement in this example. Other tissue portions of a patient may instead be included. In operation, a tip portion of the finger is inserted into casing 350 to undergo measurement. Once the finger is inserted into casing 350, optical signal 325 generated by measurement system 380 is applied to tissue 330 for measurement of a physiological parameter. In this example, optical signal 325 is applied to tissue 330 via an input optical fiber associated with optical cable 320, and optical signal 325 is detected through tissue 330 via an output optical fiber associated with optical cable 320. Optical signal 325 is coupled between the associated optical fibers and tissue 330 by tissue interface pad 310. Upon receiving optical signal 325 over the output optical fiber after propagation through tissue 330, measurement system 380 may detect and process the optical signal to determine various characteristics of the detected optical signal. Physiological parameters of the tissue and patient can then be identified based on the various characteristics of the detected optical signals.

Tissue interface pad 310 may be composed of plastic, foam, rubber, glass, metal, adhesive, or some other material, including combinations thereof. Tissue interface pad 310 includes a generally planar surface configured to interface with tissue 330 to allow for introduction of optical signals into tissue 330 and for receipt of optical signals from tissue 330. Tissue interface pad 310 also may include elements as discussed above for tissue interface pad 110, although these elements can use different configurations.

Pressure cuff 340 is configured to apply a pressure received over pressure link 341 from pressure system 390 to tissue interface pad 310 to couple at least a portion of tissue interface pad 310 to tissue 330. Pressure cuff 340 also may include elements as discussed above for pressurized volume 140, although these elements can use different configurations. The size, shape, and configuration of pressure cuff 340 can vary according to many factors. For example, properties of casing 350, tissue 330, and tissue interface pad 310 each can influence the size, shape, and configuration of pressure cuff 340, among other factors including desired pressure. Although pressure cuff 340 is shown as an inflatable pad or balloon-style pressurized volume in FIG. 3, it should be understood that a wrap-around cuff as shown in FIG. 1 may instead be employed. Pressure link 341 can comprise tubing, piping, or other pressure conduits for transferring a pressure generated by pressure system 390 to pressure cuff 340. Various pressure control and coupling elements can also be included, such as valves, pistons, couplers, thermally welded elements, or friction-fit elements.

Casing 350 is a rigid housing which seats tissue 330 for measurement. Casing 350 includes preload tension elements 352 for applying a preload pressure to tissue 330 to initially align tissue 330 in casing 350 to tissue interface pad 310 and likewise to pressure cuff 340. Three pairs of preload studs 351 are included in this example to attach each preload tension element 352 to casing 350. Preload tension elements 352 can include elastic bands, rubber cords, shock cords, fabric sleeves, springs, or other tension element to place a preload pressure on tissue 330 in casing 350. By employing preload tension elements 352, casing 350 initially adapts to different finger or toe sizes and shapes before application of a pressure by pressure cuff 340. Casing 350 also includes an angled tip to accommodate a tip of a finger or toe of a patient. As pressure cuff 340 applies a pressure, such as due to inflation, tissue 330 can move against preload tension elements 352 while maintaining alignment and contact with tissue interface pad 310. Example preloading pressure exerted on tissue 330 by preload tension elements 352 include 5-10 mmHg, to ensure adequate pressure and contact between tissue 330 and tissue interface pad 310 once pressure cuff 340 is inflated. The preload pressure typically is configured to ensure slight engagement of tissue interface pad 310 on tissue 330. An example over-pressure of the preload is 20 mmHg or greater. Other preload pressures or tensions can be applied, and preload pressure or tension can be determined based on a size of the tissue of the patient, such as a finger size. For example, a larger diameter finger may use a smaller preload tension while a smaller diameter finger may use a larger preload tension, or vice versa, to maintain a desired preload pressure of tissue 330 on tissue interface pad 310. This example illustrates casing 350 suited for a tip of a finger or toe of a patient and a smaller casing is thus employed. A larger casing is discussed in FIG. 4.

Measurement system 380 includes optical interfaces, digital processors, computer systems, microprocessors, circuitry, non-transient computer-readable media, user interfaces, or other processing devices or software systems, and may be distributed among multiple processing devices. Measurement system 380 may also include photon density wave (PDW) generation and measurement equipment, electrical to optical conversion circuitry and equipment, optical modulation equipment, and optical waveguide interface equipment. Measurement system 380 also includes laser elements such as a laser diode, solid-state laser, or other laser device, along with associated driving circuitry. Optical couplers, cabling, or attachments can be included to optically mate laser or detector elements to optical fibers of optical cable 320.

Pressure system 390 includes pressure generation, control, and monitoring equipment. Pressure system 390 can include pumps, pistons, syringes, pressure gauges, pressure sensors, user control and monitoring interfaces. Pressure system 390 can also comprise tubing, piping, or other pressure conduits for transferring a pressure generated by pressure system 390 to pressure cuff 340. Various pressure control and coupling elements can also be included, such as valves, pistons, couplers, thermally welded elements, or friction-fit elements.

FIG. 4 is a system diagram illustrating system 400 for applying optical signals to tissue 430 of a patient. System 400 includes tissue interface pad 410, optical cable 420, tissue 430, pressure cuff 440, casing 450, composite cable 460, measurement system 480, and pressure system 490. A top view and a side view of system 400 are included in FIG. 4 to highlight the various elements of system 400. It should be understood the dashed features of FIG. 4 are merely intended to highlight various elements of system 400, and are not intended to be exact wireframe representations of the elements of system 400; variations are possible.

Tissue 430 is shown as a finger of a patient undergoing measurement in this example. Other tissue portions of a patient may instead be included. In operation, a portion of the finger is inserted into casing 450 to undergo measurement. Once the finger is inserted into casing 450, optical signal 425 generated by measurement system 480 is applied to tissue 430 for measurement of a physiological parameter. In this example, optical signal 425 is applied to tissue 430 via an input optical fiber associated with optical cable 420, and optical signal 425 is detected through tissue 430 via an output optical fiber associated with optical cable 420. Optical signal 425 is coupled between the associated optical fibers and tissue 430 by tissue interface pad 410. Upon receiving optical signal 425 over the output optical fiber after propagation through tissue 430, measurement system 480 may detect and process the optical signal to determine various characteristics of the detected optical signal. Physiological parameters of the tissue and patient can then be identified based on the various characteristics of the detected optical signals.

Tissue interface pad 410 may be composed of plastic, foam, rubber, glass, metal, adhesive, or some other material, including combinations thereof. Tissue interface pad 410 includes a generally planar surface configured to interface with tissue 430 to allow for introduction of optical signals into tissue 430 and for receipt of optical signals from tissue 430. Tissue interface pad 410 also may include elements as discussed above for tissue interface pads 110 or 310, although these elements can use different configurations.

Pressure cuff 440 is configured to apply a pressure received over pressure link 441 from pressure system 490 to tissue interface pad 410 to couple at least a portion of tissue interface pad 410 to tissue 430. Pressure cuff 440 also may include elements as discussed above for pressurized volume 140 or pressure cuff 340, although these elements can use different configurations. Although pressure cuff 440 is shown as an inflatable pad or balloon-style pressurized volume in FIG. 4, it should be understood that a wrap-around cuff as shown in FIG. 1 may instead be employed. Pressure link 441 can comprise tubing, piping, or other pressure conduits for transferring a pressure generated by pressure system 490 to pressure cuff 440. Various pressure control and coupling elements can also be included, such as valves, pistons, couplers, thermally welded elements, or friction-fit elements.

Casing 450 is a rigid housing which seats tissue 430 for measurement. Casing 450 includes preload tension elements 452 for applying a preload pressure to tissue 430 to initially align tissue 430 in casing 450 to tissue interface pad 410 and likewise to pressure cuff 440. Six pairs of preload studs 451 are included in this example to attach each preload tension element 452 to casing 450. Preload tension elements 452 can include elastic bands, rubber cords, shock cords, fabric sleeves, springs, or other stretchable element to place a preload pressure on tissue 430 in casing 450. By employing preload tension elements 452, casing 450 initially adapts to different finger or toe sizes and shapes before application of a pressure by pressure cuff 440. Casing 450 includes an angled tip to accommodate a tip of a finger or toe of a patient, as well as an angled casing portion to accommodate the length of a finger or toe to allow for a more uniform preload along the length of tissue 430. Casing 450 also includes sensor box 453 to hold tissue interface pad 410 and pressure cuff 440 in casing 450 while still allowing for tissue 430 to slide into casing 450. As pressure cuff 440 applies a pressure, such as due to inflation, tissue 430 can move against preload tension elements 452 while maintaining alignment and contact with tissue interface pad 410. This example illustrates casing 450 suited for a full length of a finger or toe of a patient and a larger casing is thus employed. A smaller casing is discussed in FIG. 3.

Measurement system 480 and pressure system 490 can include similar elements as discussed above for measurement system 380 and pressure system 390 of FIG. 3. In this example, optical cable 420 and pressure link 441 are also included in a composite cable 460. Since measurement system 480 and pressure system 490 may be located remotely from tissue 430, the optical and pressure links are bundled in a loom, casing, sheath, or the like, which allows for bundling and common routing of the associated links between systems 480-490 and casing 450. Composite cable 460 can be routed generally parallel to the tissue under measurement, such as a finger of a patient.

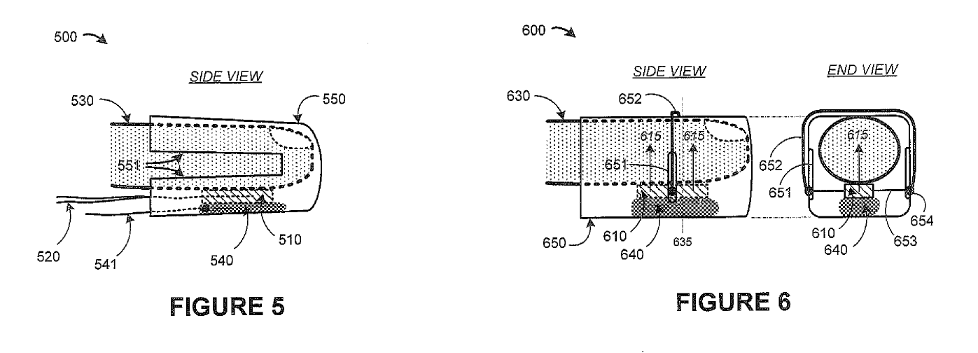

FIG. 5 is a system diagram illustrating system 500 for applying optical signals to tissue 530 of a patient. System 500 includes tissue interface pad 510, optical links 520, tissue 530, pressure pad 540, and boot 550. Associated measurement and pressure systems are omitted from FIG. 5 for clarity, but similar measurement and pressure systems as found in FIGS. 1-4 may be included. Also, optical signals propagated within tissue 530 are omitted in FIG. 5 for clarity and to highlight mechanical features of system 500. A side view of system 500 is included in FIG. 5 to highlight the various elements of system 500. It should be understood the dashed features of FIG. 5 are merely intended to highlight various elements of system 500, and are not intended to be exact wireframe representations of the elements of system 500; variations are possible.

Tissue 530 is shown as a finger of a patient undergoing measurement in this example. Other tissue portions of a patient may instead be included. In operation, a portion of the finger is inserted into boot 550 to undergo measurement. Once the finger is inserted into boot 550, optical signals are applied to tissue 530 for measurement of a physiological parameter. Optical signals are coupled between tissue 530 by tissue interface pad 510. As discussed herein, optical signals may be detected and processed to determine various characteristics of the detected optical signals. Physiological parameters of the tissue and patient can then be identified based on the various characteristics of the detected optical signals.

Tissue interface pad 510 may be composed of plastic, foam, rubber, glass, metal, adhesive, or some other material, including combinations thereof. Tissue interface pad 510 includes a generally planar surface configured to interface with tissue 530 to allow for introduction of optical signals into tissue 530 and for receipt of optical signals from tissue 530. Tissue interface pad 510 also may include elements as discussed above for tissue interface pads 110, 310, or 410, although these elements can use different configurations.

Pressure pad 540 is configured to apply a pressure received over pressure link 541 from pressure system 590 to tissue interface pad 510 to couple at least a portion of tissue interface pad 510 to tissue 530. Pressure pad 540 also may include elements as discussed above for pressurized volume 140 or pressure cuffs 340 or 440, although these elements can use different configurations. Although pressure pad 540 is shown as an inflatable pad or balloon-style pressurized volume in FIG. 5, it should be understood that a wrap-around cuff as shown in FIG. 1 may instead be employed. Pressure link 541 can comprise tubing, piping, or other pressure conduits for transferring a pressure generated by a pressure system to pressure pad 540. Various pressure control and coupling elements can also be included, such as valves, pistons, couplers, thermally welded elements, or friction-fit elements.

Boot 550 is a rigid housing which seats tissue 530 for measurement. Boot 550 includes notch element 551 for allowing for various sizes of tissue 530, such as different finger or toe sizes. Notch element 551 may be a v-groove, square notch, rounded notch, or the like. By using notch element 551, boot 550 initially flexes and adapts to different finger to toe sizes and shapes before application of a pressure by pressure pad 540. Boot 550 includes a rounded tip to accommodate a tip of a finger or toe of a patient. As pressure pad 540 applies a pressure, such as due to inflation, boot 550 maintains alignment and contact between tissue 530 and tissue interface pad 510.

FIG. 6 is a system diagram illustrating system 600 for applying optical signals to tissue 630 of a patient. System 600 includes tissue interface pad 610, tissue 630, pressure pad 640, and boot 650. Associated measurement and pressure systems are omitted from FIG. 6 for clarity, but similar measurement and pressure systems as found in FIGS. 1-4 may be included. Also, pressure links, optical links and optical signals propagated within tissue 630 are omitted in FIG. 6 for clarity and to highlight mechanical features of system 600. An end view and a side view of system 600 are included in FIG. 6 to highlight the various elements of system 600. The side view is sectioned approximately at section cut 635. It should be understood the dashed features of FIG. 6 are merely intended to highlight various elements of system 600, and are not intended to be exact wireframe representations of the elements of system 600; variations are possible.

Tissue 630 is shown as a finger of a patient undergoing measurement in this example. Other tissue portions of a patient may instead be included. In operation, a portion of the finger is inserted into casing 650 to undergo measurement. Once the finger is inserted into casing 650, optical signals are applied to tissue 630 for measurement of a physiological parameter. Optical signals are coupled between tissue 630 by tissue interface pad 610. As discussed herein, optical signals may be detected and processed to determine various characteristics of the detected optical signals. Physiological parameters of the tissue and patient can then be identified based on the various characteristics of the detected optical signals.

Tissue interface pad 610 may be composed of plastic, foam, rubber, glass, metal, adhesive, or some other material, including combinations thereof. Tissue interface pad 610 includes a generally planar surface configured to interface with tissue 630 to allow for introduction of optical signals into tissue 630 and for receipt of optical signals from tissue 630. Tissue interface pad 610 also may include elements as discussed above for tissue interface pads 110, 310, 410, or 510, although these elements can use different configurations.

Pressure pad 640 is configured to apply a pressure to tissue interface pad 610 to couple at least a portion of tissue interface pad 610 to tissue 630. Pressure pad 640 also may include elements as discussed above for pressurized volume 140 or pressure cuffs or pads 340, 440, or 540, although these elements can use different configurations. Although pressure pad 640 is shown as an inflatable pad or balloon-style pressurized volume in FIG. 6, it should be understood that a wrap-around cuff as shown in FIG. 1 may instead be employed.

Boot 650 is a rigid housing which seats tissue 630 for measurement. Boot 650 includes adjustable preload elements 651-654. Tissue interface pad 610 is coupled to shaft 653 which fits into slots 651. The ends of shaft 653 include rivets 654 to allow shaft 653 to slide within slots 651 while preventing escape of shaft 653 and providing side-to-side alignment of tissue interface pad 610 within casing 650. Coupled to rivets 654 is optional tension member 652. Tension member 652 can include elastic bands, rubber cords, shock cords, fabric sleeves, springs, or other stretchable element to place a preload pressure 615 on tissue interface pad 610 and likewise onto tissue 630 held in casing 650. Since shaft 653 is coupled to tissue interface pad 610, the tension provided by tension member 652 to rivets 654 will cause shaft 653 to slide in slots 651 and automatically adjust to different sizes of tissue 630. After alignment and adjustment via adjustable preload elements 651-654 a pressure can be applied via pressure pad 640. The various elements of adjustable preload elements 651-654 can be composed of metal, plastic, wood, composite material, rubber, or other material, including combinations thereof.

In the examples discussed herein, the pressurized volumes are typically configured to apply a pressure on a tissue interface pad to maintain a desired contact pressure of a tissue interface pad on tissue undergoing measurement. This pressure is adjusted to maintain a desired pressure and optical signal quality in the tissue due to the effect of varying tissue sizes, skin conditions, ambient conditions, or other factors which may affect the measurement process. To ensure the desired contact pressure is maintained, a threshold signal quality of optical signals in the tissue of a patient can be monitored, such as by measurement systems 180, 380, or 480. For example, a detected portion of the optical signals can be monitored to determine if a threshold signal quality is met. The signal quality can include a signal-to-noise ratio, magnitude of a signal component, or other signal quality metric. In some examples, the optical signals can include detected tissue parameters, such as pulsatile signal components due to the pulse of the patient, or other tissue parameters identified after propagating the optical signals through tissue. The threshold signal quality can thus include a threshold level of the pulsatile signal detected from the optical signals through the tissue. The threshold signal quality can include any threshold tissue parameter. A measurement system can also be configured to identify the detected signal quality of the propagated optical signals and determine a target pressure for the pressurized volume based on the detected signal quality and a desired signal quality. For example, if the measurement system determines that the detected signal quality is too poor or below a low threshold level, then the pressure applied by a pressure system to the pressurized volume can be increased. This increase in pressure forces the associated tissue interface pad more tightly against the tissue and can provide for a higher intensity of the propagated signal in the tissue. Likewise, if the measurement system determines that the detected signal quality is above a high threshold level, then the pressure applied by a pressure system to the pressurized volume can be decreased. This decrease in pressure forces the associated tissue interface pad less tightly against the tissue and can provide for a lower intensity of the propagated signal in the tissue. A pressure may be too high if the optical signal indicates clipping from too high of detected signal intensity or if the signal exceeds a dynamic range of signal processing circuitry. It should be understood that the measurement system and associated pressure system can be configured to communicate to adjust the applied pressure based on the optical signal quality.

In order to determine the desired pressure to be applied to the pressurized volume, a pressure system can be configured to apply a range of pressures to the pressurized volume. A target pressure can be identified when monitored optical signal quality factors fall within a threshold range, as discussed above. The pressure system or measurement system can then identify the target pressure for the pressurized volume to obtain the desired signal quality and apply the target pressure as the pressure to the pressurized volume.

Although a pressurized volume, such as a pressure cuff or pad, has been discussed herein, other configurations can be employed. For example, a thermally sensitive spring element can be employed to apply an adjustable pressure to a tissue interface pad. An electronic heating element can be coupled to the thermally sensitive spring to modify a spring constant, and likewise an applied pressure, based on a heat applied by the heating element to the spring. Thus, an adjustable pressure can be applied by the adjustable spring element instead of an inflatable volume. Another configuration includes a servo-gear mechanism using a lever to apply an adjustable pressure to a tissue interface pad.

The included descriptions and drawings depict specific embodiments to teach those skilled in the art how to make and use the best mode. For the purpose of teaching inventive principles, some conventional aspects have been simplified or omitted. Those skilled in the art will appreciate variations from these embodiments that fall within the scope of the invention. Those skilled in the art will also appreciate that the features described above can be combined in various ways to form multiple embodiments. As a result, the invention is not limited to the specific embodiments described above, but only by the claims and their equivalents.

What is claimed is:

1. A tissue interface system for applying optical signals to tissue of a patient, the tissue interface system comprising:
a tissue interface pad configured to apply the optical signals carried by at least one optical source into the tissue; and
a pressurized volume configured to apply pressure to the tissue interface pad to couple a portion of the tissue interface pad to the tissue.

2. The tissue interface system of claim 1, wherein the pressurized volume is configured to apply the pressure to maintain a threshold signal quality of a detected portion of the optical signals.

3. The tissue interface system of claim 2, wherein the threshold signal quality comprises a threshold level of a tissue parameter detected from the optical signals through the tissue.

4. The tissue interface system of claim 1, further comprising:
a measurement system configured to identify a detected signal quality of the optical signals and determine a target pressure for the pressurized volume based on the detected signal quality and a desired signal quality.

5. The tissue interface system of claim 4, further comprising:
a pressure system configured to apply a range of pressures to the pressurized volume to identify the target pressure for the pressurized volume to obtain the desired signal quality and apply the target pressure as the pressure to the pressurized volume.

6. The tissue interface system of claim 1, wherein the pressurized volume comprises a pressure cuff configured to receive and apply the pressure provided from a pressure system to the portion of the pressure interface pad.

7. The tissue interface system of claim 1, further comprising:
a casing configured to align a portion of the tissue with the tissue interface pad and the pressurized volume.

8. The tissue interface system of claim 7, comprising:
the casing comprising at least one tension member configured to hold the portion of the tissue against the tissue interface pad and the pressurized volume at a preload pressure, wherein the pressurized volume is configured to apply the pressure to the portion of the tissue interface pad after application of the preload pressure to the tissue.

9. The tissue interface system of claim 8, wherein the tissue comprises a finger of the patient, and the preload pressure is determined based on a size of the finger of the patient.

10. The tissue interface system of claim 1, wherein the tissue comprises a finger of the patient, wherein the tissue interface pad is coupled to an optical cable comprising at least one optical fiber as the optical source, wherein the pressurized volume is coupled to a pressure tube carrying the pressure, and wherein the optical cable and the pressure tube are aligned generally parallel to each other and to the finger of the patient.

11. A method for applying optical signals to tissue of a patient, the method comprising:
applying the optical signals carried by at least one optical source into the tissue by a tissue interface pad; and
applying a pressure from a pressurized volume to the tissue interface pad to couple a portion of the tissue interface pad to the tissue.

12. The method of claim 11, wherein applying the pressure comprises applying the pressure to maintain a threshold signal quality of a detected portion of the optical signals.

13. The method of claim 12, wherein the threshold signal quality comprises a threshold level of a tissue parameter detected from the optical signals through the tissue.

14. The method of claim 11, further comprising:
identifying a detected signal quality of the optical signals and determining a target pressure for the pressurized volume based on the detected signal quality and a desired signal quality.

15. The method of claim 14, further comprising:
applying a range of pressures to the pressurized volume to determine the target pressure for the pressurized volume to obtain the desired signal quality, and applying the target pressure as the pressure to the pressurized volume.

16. The method of claim 11, wherein the pressurized volume comprises a pressure cuff for receiving and applying the pressure provided from a pressure system to the portion of the pressure interface pad.

17. The method of claim 11, further comprising:
aligning via a casing a portion of the tissue with the tissue interface pad and the pressurized volume.

18. The method of claim 17, wherein the casing comprises at least one tension member for holding the portion of the tissue against the tissue interface pad and the pressurized volume at a preload pressure; and wherein applying the pressure from the pressurized volume to the tissue interface pad comprises applying the pressure to the portion of the tissue interface pad after application of the preload pressure to the tissue.

19. The method of claim 18, wherein the tissue comprises a finger of the patient, and the preload pressure is determined based on a size of the finger of the patient.

20. The method of claim 11, wherein the tissue comprises a finger of the patient, wherein the tissue interface pad is coupled to an optical cable comprising at least one optical fiber as the optical source, wherein the pressurized volume is coupled to a pressure tube carrying the pressure, and wherein the optical cable and the pressure tube are aligned generally parallel to each other and to the finger of the patient.
